(19) United States
(12) Patent Application Publication
(10) Pub. No.: US 2007/0071777 A1
(43) Pub. Date: Mar. 29, 2007

(54) Oil emulsion for postnatal hormone substitution

(76) Inventors: Sonja Bromer, Melsungen (DE); Jorg Nehne, Guxhagen (DE); Frank Pohlandt, Ulm (DE)

Correspondence Address: CHRISTIE, PARKER & HALE, LLP, PO BOX 7068, PASADENA, CA, 91109-7068, US

(21) Appl. No.: 10/561,631

(22) PCT Filed: Jun. 15, 2004
(86) PCT No.: PCT/EP04/51114

§ 371 Date: Oct. 31, 2006

(30) Foreign Application Priority Data
Jun. 18, 2003 (EP) 03013790.5

(51) Int. Cl. A61K 31/57; A61K 31/56; A61K 9/00

(52) U.S. Cl. 424/400; 514/170

(57) ABSTRACT

The invention relates to a process for the preparation of hormone-containing oil emulsions (lipid emulsions), an isotonic oil emulsion obtainable by such process, and the use of the emulsion according to the invention for the preparation of a medicament for intravenous administration, especially for postnatal hormone substitution in premature babies and for the treatment of neurological damage after strokes.

The invention relates to a process for the preparation of hormone-containing oil emulsions (lipid emulsions), an isotonic oil emulsion obtainable by such process, and the use of the emulsion according to the invention for the preparation of a medicament for intravenous administration, especially for postnatal hormone substitution in premature babies and for the treatment of neurological damage after strokes.

### TECHNICAL BACKGROUND OF THE INVENTION

During pregnancy, the plasma levels of 17-β-estradiol (an estrogen) and progesterone (a progestagen) increase up to 100 fold. This enhanced synthesis of estrogen and progesterone serves for maintaining pregnancy, inter alia. From examinations of cord blood at different times during pregnancy, it can be seen that the fetus is also exposed to these high plasma levels. There are clear indications of the fact that the fetal development of various organs, such as the lungs, bones and brain, depends on estrogen and progesterone. Full term childbirth occurs after 40 weeks of pregnancy, after which a rapid decrease of estradiol and progesterone occurs in both the newborn and the mother. This decrease of the hormone levels is presumably co-responsible for the depression from which women frequently suffer after childbirth.

In the Federal Republic of Germany, from 6 to 7% of all newborns are premature babies, i.e., the day of birth is before the end of the 37th week of pregnancy. Thus, the rapid decrease of the plasma levels of estradiol and progesterone occurs at an earlier time during fetal development, which might have consequences for the still immature organs. Even today, immature lung function in extremely small premature babies (below 1000 g birth weight) is still a frequent cause of newborn mortality. Follow-up studies on former premature babies also show that their neurological development is adversely affected on a long-term basis.

Studies on 30 premature babies made by Trotter et al. at the children's hospital of the University of Ulm (published in J. Clin. Endocrinol. Metab. 84, 4531-4355 (1999)) show that premature babies can benefit from a maintenance of the high plasma levels of estradiol and progesterone. Premature babies were continuously intravenously administered a diluted lipid emulsion which contained the hormones 17β-estradiol and progesterone in amounts sufficient to maintain the plasma levels as found in the womb. It was found that the premature babies exhibited a better median bone mineralization. In addition, it could be observed that the additional administration of oxygen was less frequently necessary in hormone-treated premature babies at the age of 28 days. This observation can be attributed to a better lung maturation. The intravenous administration of the sexual hormones was effected over an average of 3 weeks.

In the above described study, the hormones estrogen and progesterone in an alcoholic solution were added to the lipid emulsion which was manufactured by the company Pharmacia & Upjohn and sold under the trade name of Intralipid®, and administered in this form. The oil phase of Intralipid® consists of 100% soybean oil in the form of long-chain triglycerides.

More exact chemical analyses show that there are considerable uncertainties in the admixing of the sexual hormones with Intralipid®. For example, it is unclear whether the mixture of the steroids in Intralipid® is physically stable at all. However, experiments with radioactively labeled hormones showed that a considerable fraction of the hormones to be applied is absorbed at the surface of the infusion systems and feed ducts. This necessarily results in problems and uncertainties with respect to the availability of the intravenously administered hormones to the infant.

In addition, in the course of the above described studies it was found that the administration of relatively large amounts of hormone-containing oil emulsion resulted in comparatively low hormone levels in the premature Baby's blood. Therefore, to achieve the desired serum levels, relatively high amounts of oil emulsions have had to be administered to the premature babies to date. These high amounts resulted in an undesirable oil and liquid load on the premature baby. Moreover, the administration of alcoholic compositions to premature babies is not a preferred treatment method.

In addition, follow-up examination at the corrected age of 15 months of the premature babies treated in the pilot study reveals a positive influence on the neurological development (published in J. Clin. Endocrinol. Metab. 86, 601-603 (2001)).

A transepidermal treatment with the corresponding hormones is possible in principle, but can be started only 2 to 3 weeks after birth for developmental reasons.

The presently available results show a positive influence of the hormone treatment on the target criteria, i.e., lung maturation and development, neurological development and bone mineralization, i.e., an overall improvement of the premature babies' maturation. In addition, the parenterally administered lipid emulsion also serves mere nutrition purposes by supplying oil in an intravenously tolerable dosage form to the premature organism for which enteral food supply is often difficult.

Further, Alkayed et al. in Stroke 31, 161 (2003), describe the positive influence of subcutaneously administered estrogens and progesterones on the condition of stroke patients.

Therefore, the object of the invention is to provide parenterally administrable oil emulsions which result in as high as possible an enrichment of the hormones (availability) in the premature baby's blood with a minimum oil and volume load, in contrast to the emulsions described in the prior art.

Surprisingly, it has been found that the parenteral administration of an oil emulsion containing estradiol and progesterone in the preparation of which the hormones are dissolved in the oil phase before the emulsification results in clearly higher serum levels as compared to the administration of emulsions to which the hormones are directly added.

### DESCRIPTION OF THE FIGURES

FIG. 1 describes the estradiol plasma level at the individual measuring times (day 1, 3, 7, 14) in the premature babies treated with the different emulsions.

FIG. 2 describes the progesterone plasma level at the individual measuring times (day 1, 3, 7, 14) in the premature babies treated with the different emulsions.

### DESCRIPTION OF THE INVENTION

The present invention relates to a process for the preparation of isotonic oil emulsions containing estrogen and progestagen for parenteral, preferably intravenous, administration comprising the steps of:
(A) dissolving at least one of the hormones estrogen and progestagen in an oil phase; and
(B) emulsifying the oil phase in the aqueous phase;
   in the presence of an emulsifier.

Another embodiment of the invention relates to a hormone-containing isotonic oil emulsion for intravenous application which can be obtained by the above process. In a preferred embodiment, progestagen and estrogen are in a ratio of from 2:1 to 200:1 in the emulsion.

The o/w emulsions according to the invention are suitable for parenteral, especially intravenous, administration. Accordingly, a third embodiment of the invention relates to the use of the above isotonic oil emulsion for intravenous administration, especially for postnatal hormone substitution in premature babies.

In connection with the present invention, the terms “oil” and “lipid” have the same meaning and are therefore used interchangeably. This group of substances includes, in particular, triglycerides, partial glycerides and fatty acid residues as well as their mixtures.

In particular, the oil emulsions according to the invention are suitable for processes for the preparation of medicaments for the parenteral administration of estrogens and progestagens, preferably for postnatal hormone substitution in premature babies.

Further embodiments of the invention can be seen from the dependent claims.

The postnatal substitution of progestagen and estrogen aims at the maintenance of intra-uterine plasma levels (plasma level in utero). In order to keep the deviation from the intra-uterine plasma levels as low as possible after birth, it is desirable that such levels are reached as quickly as possible after the beginning of the substitution.

Hormone substitution is not restricted to premature human babies, but may also be applied to animals, preferably mammals.

The oil emulsions according to the invention have a clearly improved availability of the hormones contained therein as compared to that of an oil emulsion of the prior art.

As compared to prior art emulsions, the oil emulsion according to the invention results in a faster increase of the hormone level and in a higher estrogen end concentration while the volume and oil loads are lower.

In addition, the oil emulsions according to the invention have an improved stability as compared to the alcoholic hormone solutions of the prior art.

The critical step of the process for the preparation of the oil emulsions according to the invention is the dissolution of the hormones estradiol and progesterone in the oil phase before the oil phase is emulsified with the aqueous phase. Basically, both hormones are dissolved in the oil phase. In another embodiment according to the invention, only one of the hormones, preferably an estrogen, is dissolved in the oil phase while the progestagen is added to the aqueous phase and/or to the finished emulsion.

The hormones used in connection with the present invention are those which also occur in utero. A distinction is made, in particular, between the follicular hormones (estrogens) and the hormones of the yellow body (progestagens).

The follicular hormones which are important in connection with the present invention are estrone, 17β-estradiol and estriol and their derivatives. Due to its high estrogenic activity, 17β-estradiol is of particular importance for the present invention.

The progestagens used in connection with the present invention are pregnelonone, progesterone, medroxyprogesterone and their pharmaceutically acceptable derivatives, progesterone being used preferably in connection with the present invention.

One embodiment of the invention relates to the combination of estrone with pregnelonone and/or progesterone, another to the combination of estriol with pregnelonone and/or progesterone. An alternative, particularly preferred embodiment relates to the combination of 17β-estradiol and/or pregnelonone and/or progesterone, especially with progesterone. In both alternatives, medroxyprogesterone may be additionally contained, or medroxyprogesterone may be substituted for pregnelonone and/or progesterone. Thus, more than two hormones may also be combined according to the invention.

The lipid emulsions according to the invention have hormone concentrations which, when accordingly employed, result in plasma levels in the premature baby as would have been to be expected in the womb. Consequently, the lipid emulsion according to the invention comprises between 0.005 and 0.5% by weight, preferably between 0.01 and 0.2% by weight, more preferably between 0.05 and 0.1% by weight, of at least one estrogen, and between 0.05 and 5% by weight, preferably between 0.1 and 2% by weight, more preferably between 0.5 and 1% by weight, of at least one progestagen, based on the total composition (parent emulsion).

The ratio of progestagen to estrogen in the emulsion is from 2:1 to 200:1, preferably from 5:1 to 50:1, more preferably from 10:1 to 20:1.

For a better dosing of the oil emulsions, the parent emulsions can be diluted, if necessary, with an appropriate amount of water, preferably with up to the fourfold amount of water.

The lipid emulsions according to the invention are preferably prepared from oils of vegetable origin (e.g., safflower oil or soybean oil) and/or MCT and/or oils of animal origin. Therefore, they can contain vegetable oil and/or medium-chain triglycerides (MCT) and/or oils of marine origin (e.g., fish oils). Such lipid emulsions are known to the skilled person from the prior art.

Vegetable oils and especially the oils of soybean and safflower are characterized by a high content of polyunsaturated fatty acids of the ω-6 series (predominantly linolic acid, 18:2 ω-6), while their content of ω-3 fatty acids (virtually exclusively as α-linolenic acid, 18:3 ω-3) is low.

Medium-chain triglycerides (MCT) have a chain length of from C₆ to C₁₄, a chain length of from C₈ to C₁₀ being particularly preferred.

The medium-chain triglycerides (MCT) administered with the oil emulsions predominantly serve as an energy source. Medium-chain triglycerides do not contain any unsaturated fatty acids at all, and they thus contain neither ω-6 nor ω-3 fatty acids.

The fish oils obtained from cold-water fish are characterized by a high content of polyunsaturated fatty acids (mainly eicosapentaenic acid, EPA, 20:5 ω-3 and docosahexaenic acid, DHA, 22:6 ω-3), while their content of ω-6 fatty acids is low. Suitable fish oils are those, for example, which are obtained from cold-water fish industrially in large amounts. Fish oils generally contain triglycerides of fatty acids having from 12 to 22 carbon atoms. Particularly preferred are highly purified fish oil concentrates which are obtained, for example, from sardine oil, salmon oil, herring oil and/or mackerel oil.

Therefore, one embodiment according to the invention relates to an oil emulsion based on vegetable oil and/or MCT. This emulsion may optionally contain fish oil.

The content of vegetable oil in the oil composition according to the invention is at least from 50 to 100% by weight, preferably from 70 to 100% by weight, more preferably from 90 to 100% by weight, based on the oil composition.

The beneficial effect of unsaturated fatty acids, especially those of the ω-3 series, is known to the skilled person and has been described, for example, in EP-A-0 311 091 and DE-A-19648566. Also in connection with the present invention, the use of oils which are rich in unsaturated fatty acids may be advantageous.

The total oil content of the parent emulsion is between 1% by weight and 30% by weight, preferably between 10% by weight and 20% by weight, based on the aqueous oil emulsion.

In addition to distilled water, the isotonic oil emulsion may also contain the usual auxiliary agents and/or additives, such as emulsifiers, co-emulsifiers, stabilizers, antioxidants and isotonizing additives.

Physiologically acceptable emulsifiers, such as phospholipids of animal or vegetable origin, are used as the emulsifiers. Particularly preferred are purified lecithins, especially egg lecithin or fractions thereof or the corresponding phosphatides. The content of emulsifier is from 0.6 to 1.5% by weight, preferably 1.2% by weight, based on the total emulsion.

Further, alkali salts of long chain C₁₆ to C₂₀ fatty acids may be used as co-emulsifiers. Their sodium salts are particularly preferred. The co-emulsifiers are employed in a concentration of from 0.005 to 0.1% by weight, preferably from 0.01 to 0.5% by weight, based on the total emulsion.

For stabilization and isotonization, the emulsion according to the invention may contain from 1.0 to 8% by weight, preferably from 2.0 to 6.0% by weight, more preferably from 2.2 to 2.6% by weight, of a stabilizing or isotonizing additive, for example, a polyhydric alcohol. In this connection, glycerol, glucose or xylitol are preferred, glycerol being particularly preferred.

As antioxidants and thus for protection from peroxide formation, the oil emulsion according to the invention may contain tocopherols or physiologically acceptable tocopherol esters, e.g., alpha-tocopherol acetate, in an amount of from 10 to 1000 mg, preferably from 25 to 200 mg, based on 100 g of oil.

Of course, no additives are used which have any undesirable side effects or cause intolerances. In particular, fructose and sorbitol are suspected to cause intolerances and are therefore unsuitable in connection with the present invention. Further, the oil emulsions according to the invention do not contain any preservatives, such as benzyl alcohol.

The oil emulsions according to the invention are always oil-in-water (o/w) emulsions in which the outer, continuous phase consists of distilled water adapted for parental use.

The oil emulsion advantageously has a pH value of from 6.0 to 9.0, preferably from 6.5 to 8.5.

The isotonic aqueous lipid emulsions according to the inventions can be prepared by known methods. The usual approach is to mix the oils, the emulsifier and other auxiliary agents and additives at first and then to add water while dispersing. The water may optionally contain further water-soluble components (e.g., glycerol). The thus obtained emulsion still has droplet sizes of about 10 μm. The average droplet size of the emulsion must be reduced further by a further homogenization, e.g., by using a high-pressure homogenizer. Preferred for parenteral, especially intravenous, application are droplet sizes with a mean particle diameter of from 0.5 μm to 150 nm, more preferably from 1 μm to 100 nm. In addition, the solutions are to be sterilizable and have a storage stability of at least 18 months.

In addition to the use of the isotonic oil emulsions according to the invention for the parenteral, especially intravenous, administration of estrogen and progestagen and, in an alternative embodiment, for the preparation of a medicament for this purpose, it has been found that the oil emulsions according to the invention reduce the dying of nerve cells in the brain of humans and animals, preferably mammals, and therefore may also be employed for the preparation of medicaments for the treatment of neurological damage after strokes (apoplexy). The oil emulsions according to the invention may also be applied for prevention. In such cases, oral administration of the oil emulsions is to be preferred.

The isotonic oil emulsion according to the invention may be employed in methods for hormone substitution in premature babies as well as for the treatment of neurological damage after strokes.

The invention is illustrated by the following Examples, but without being limited thereto.

### EXAMPLES

### Preparation of a Hormone-Containing Oil Emulsion

The hormones estradiol and progesterone are dissolved in the oil heated at about 70° C. under a nitrogen inert atmosphere, optionally with the addition of tocopherol (=solution I). The emulsifier (=phospholipids from egg) is dispersed in an aqueous glycerol solution by means of an Ultra-Terrux® cell homogenizer (Jahnke & Kunkle) (=component II). Solution I is added to component II by using an Ultra-Terrux® cell homogenizer. The pH value of the resulting o/w emulsion is adjusted to about 8.5 by adding sodium oleate, followed by homogenization in a high-pressure homogenizer under at least 400 kg/cm².

After being filled into glass ampoules of suitable quality, the emulsion is heat-sterilized by known methods. A sterile and stable o/w emulsion with lipid droplets having an average oil droplet size of less than 0.5 μm and a storage stability of at least 18 months results.

TABLE 1

| | Preparation Example | 1 | | 2 | | 3 | | 4 | |
|---|---|---|---|---|---|---|---|---|---|
| I. | Estradiol hemihydrate | 0.66 | g | 0.66 | g | 0.60 | g | 0.60 | g |
| | Progesterone | 6.00 | g | 6.00 | g | 6.00 | g | 6.00 | g |
| | Medium-chain triglycerides | — | | 100 | g | 100 | g | 200 | g |
| | Purified soybean oil | 200 | g | 100 | g | 80 | g | — | |
| | Highly purified fish oil | — | | — | | 20 | g | — | |
| | α-Tocopherol | — | | — | | 200 | mg | — | |
| II. | Purified phospholipids from: | 12 | g egg | 12 | g egg | 12 | g egg | 12 | g egg |
| | Glycerol | 25 | g | 25 | g | 25 | g | 25 | g |
| | Water for injection | ad 1 liter | | ad 1 liter | | ad 1 liter | | ad 1 liter | |
| | Sodium oleate | 0.3 | g | 0.3 | g | 0.25 | g | 0.25 | g |

### Application of a Hormone-Containing Oil Emulsion

The group of patients who were treated with a hormone emulsion of the prior art consisted of 12 patients (premature babies of <29 weeks of pregnancy, birth weight below 1000 g). They were treated with a hormone emulsion consisting of 20% by weight of Intralipid® (Pharmacia & Upjohn, Germany) diluted with isotonic saline to 5% oil content, admixed with an ethanolic solution of 0.15 mg/ml crystalline 17β-estradiol and 1.4 mg/ml progesterone,

The initial feeding was continuous and intravenous (i.v.) at 15 ml/kg/day. Both the hormone content of the emulsion and the amount of liquid could be varied. This resulted in maximum liquid loads of 25.8 ml/kg/day, median 18.8.

In a currently performed randomized double-blind study relating to the influence of hormone substitution on additional oxygen demand at the age of 28 days as the target criterion, 78 premature babies have already been recruited. In this study, the hormone emulsion according to the invention (according to Preparation Example 1, diluted with water for injection to an oil content of 5%) or a placebo (hormone-free emulsion, oil content 5%) was employed. Results from 52 patients relating to the estradiol and progesterone plasma levels achieved are already available. Without unblinding, it can be derived from the plasma levels that 25 patients were treated with a hormone-containing solution (verum group). All patients were continuously administered i.v. 15 ml/kg/day of the emulsion according to the invention. From this value, a mean hormone dosage of 2.47 mg/kg/day of estradiol and 22.5 mg/kg/day of progesterone can be derived.

The plasma levels of estradiol and progesterone on day of life 1 (24 hours), 3, 7 and 14 under continuing hormone supply were determined in both groups. The results of these studies are shown in the following Table:

TABLE 2

| | Prior art emulsion | | Emulsion according to the invention | | |
|---|---|---|---|---|---|
| | Dosage | Plasma level | Dosage | Plasma level | |
| Hormone | (mg/kg/day) | (ng/ml)*⁾ | (mg/kg/day) | (ng/ml)*⁾ | P |
| Estradiol | 2.23 | 2.622 | 2.48 | 4.270 | 0.00038 |
| Progesterone | 20.23 | 286 | 22.50 | 292 | 0.153 |

*⁾median

Table 1 shows the median dosage of estradiol and progesterone until day of life 14 for the premature babies of both groups. Both emulsions resulted in a similarly high hormone supply. While the emulsion according to the invention was fed constantly with 15 ml/kg/day, the emulsion of the prior art was administered as a median of 18.8 ml/kg/day (min-max: 11.4 to 25.8 ml/kg/day).

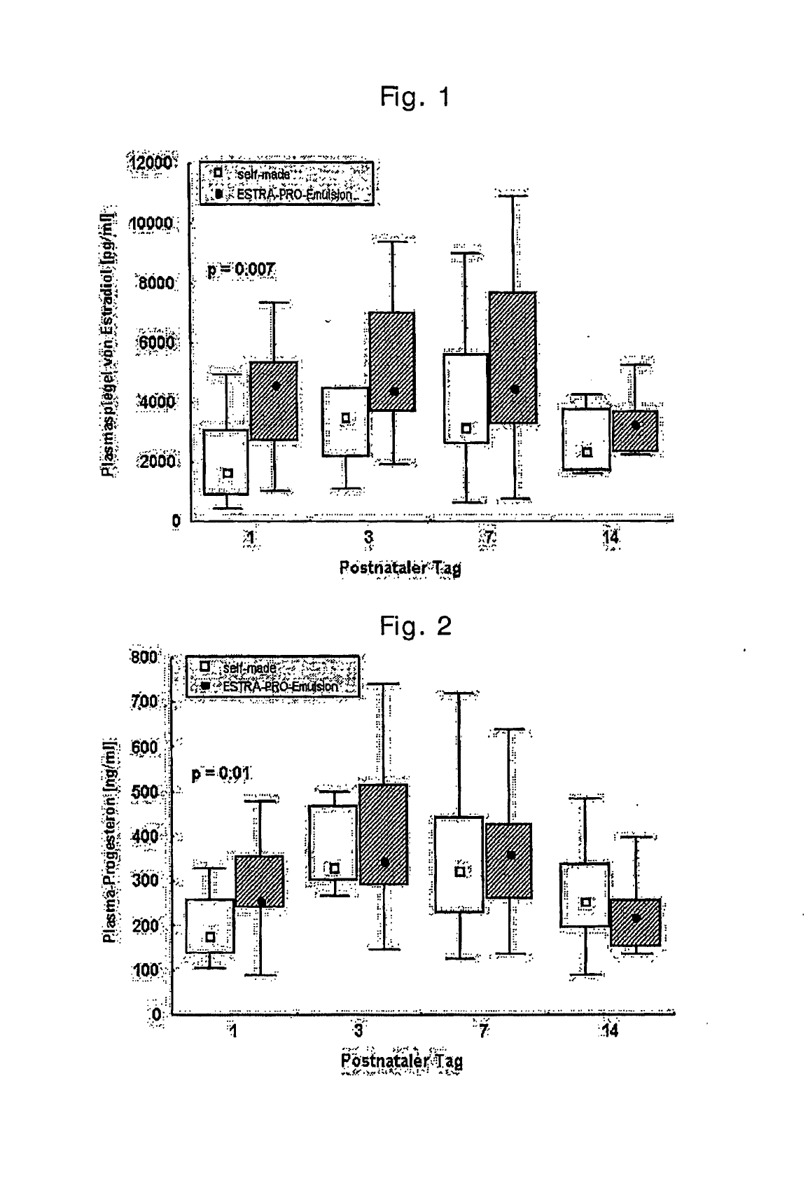

With the emulsion according to the invention, significantly higher plasma levels for estradiol could be achieved in the premature babies (p=0.00038). However, no significant difference was found in the progesterone plasma levels, when the complete period of 14 days is considered. The plasma concentrations in the premature babies treated with the different emulsions were compared with each other at the individual measuring times (day 1, 3, 7, 14) (FIGS. 1 and 2). It was found that significantly higher plasma levels were achieved with an emulsion according to the invention after 24 hours for both estradiol and progesterone. The median plasma levels of estradiol achieved on day 3, 7 and 14 with the latter emulsion were always above the values achieved with the prior art emulsion. The lower limit of the sought plasma levels of estradiol (2000 pg/ml) and progesterone (300 ng/ml) was achieved or exceeded with the emulsion according to the invention in 91% and 46% of all cases, respectively, while with the prior art emulsion, it was achieved or exceeded in only 64% and 43% of all cases, respectively.

Already after 24 hours, the plasma levels of both hormones were significantly higher for the substitution with the emulsion according to the invention as compared to the prior art emulsion, which is a progress towards the aim of reaching the plasma levels found in the womb as quickly as possible.

To conclude, the available data indicate an improved availability of estradiol and progesterone as measured by the plasma levels reached in premature babies. Thus, the emulsion according to the invention is more suitable for application with premature babies as compared to the corresponding prior art emulsion which was prepared by mixing the hormones with the finished emulsion.

1. A process for the preparation of isotonic oil emulsions containing estrogen a and progestagen for intravenous administration, comprising the steps of:
(A) dissolving at least one of the hormones estrogen and progestagen in an oil phase; and
(B) emulsifying the oil phase in the aqueous phase;
in the presence of an emulsifier.

2. A hormone-containing isotonic oil emulsion for intravenous administration comprising progestagens and estrogens, obtainable by the process according to claim 1.

3. The hormone-containing isotonic oil emulsion for intravenous administration according to claim 2, comprising progestagens and estrogens in a ratio of from 2:1 to 200:1.

4. The oil emulsion according to claim 2 or 3, characterized in that the emulsion contains from 0.005 to 0.5% by weight of an estrogen and from 0.05 to 5% by weight of a progestagen, based on the total composition.

5. The oil emulsion according to any of claims 2 to 4, characterized in that said estrogen is estradiol and said progestagen is progesterone.

6. The oil emulsion according to any of claims 2 to 5, characterized in that said oil phase comprises medium chain triglycerides (MCT) having a chain length of from 6 to 12, preferably from 8 to 10, carbon atoms.

7. The oil emulsion according to any of claims 2 to 6, characterized by containing up to 1.5% by weight of the emulsifier, based on the total composition.

8. Use of the isotonic oil emulsion according to any of claims 2 to 7 for preparing a medicament for intravenous administration of estrogen and progestagen for postnatal hormone substitution in premature babies.

9. Use of the isotonic oil emulsion according to any of claims 2 to 7 for preparing a medicament for the treatment of neurological damage after strokes.

10. A process for hormone substitution in premature babies by using the isotonic oil emulsion according to any of claims 2 to 7.

11. A process for the treatment of neurological damage after strokes by using the isotonic oil emulsion according to any of claims 2 to 7.
